(19) United States
(12) Patent Application Publication
(10) Pub. No.: US 2026/0191571 A1
(43) Pub. Date: Jul. 9, 2026

(54) LINEAR RATCHET BAR FOR PECTUS EXCAVATUM REPAIR

(71) Applicants: Brigham Young University, Provo, UT (US); Children's Hospital Medical Center, Cincinnati, OH (US)

(72) Inventors: Brandon Sargent, Provo, UT (US); Corinne Jackson, Pleasanton, CA (US); Larry L. Howell, Orem, UT (US); Bridget Beatson, Ridgefield, WA (US); Victor Garcia, Cincinnati, OH (US)

(21) Appl. No.: 19/439,217

(22) Filed: Jan. 2, 2026

Related U.S. Application Data
(60) Provisional application No. 63/741,673, filed on Jan. 3, 2025.

(51) Int. Cl. A61B 17/80

(52) U.S. Cl. CPC A61B 17/8076

(57) ABSTRACT

An implantable medical device, illustratively an arcuate bar with ratcheting pawl arms, for the gradual correction of pectus excavatum in patients.

### CROSS-REFERENCE TO RELATED APPLICATION

The present application claims priority to U.S. Provisional Patent Application Ser. No. 63/741,673, filed Jan. 3, 2025, the disclosure of which is expressly incorporated herein by reference.

### BACKGROUND AND SUMMARY OF THE DISCLOSURE

The present application relates to an implantable medical device including a ratcheting mechanism and, more particularly, to linearly ratcheting bar for use in pectus excavatum repair.

Compliant mechanisms have been favored over traditional rigid-link mechanisms in some engineering applications due to their low part count, stored strain energy, and simplicity/reliability. Such mechanisms offer potential for increased performance in medical applications, where patient safety is a priority, and where accessing a device after initial placement may be difficult, dangerous, and/or painful. Because of their stored strain energy, compliant mechanisms have the potential to perform their intended function in vivo, with little to no additional input from health professionals, greatly reducing risk and increasing patient satisfaction. They can be used in situations where self-correction (correction without any outside adjustment once the device is inserted) is desired or necessary. These benefits make compliant mechanisms an attractive alternative to correct a pectus excavatum deformity.

A phenomenon often called de-stiffening the chest wall has been observed in patients who have pectus carinatum (PC), or pigeon chest. PC is characterized by a deformed sternum that pushes outward, away from the internal organs in the chest cavity. PC is typically corrected through the use of external, wearable bracing devices. Before the correction process for PC begins, the chest wall has an in initial stiffness that opposes any displacement (like a coil spring). The phenomenon is observed once a constant force is applied to the chest wall by the brace; gradually and over time, the stiffness of the chest wall decreases, and the initial constant force then produces greater displacement. This phenomenon resembles stress relaxation in engineering materials.

The device of the present disclosure suggests that the chest wall de-stiffening phenomenon observed in PC patients will hold true for pectus excavatum patients. Pectus excavatum (PE), or funnel chest, is a deformity of the chest wall, characterized by a deformed sternum that typically produces a fist-sized depression in the chest cavity. It is the most common chest wall deformity, with a rate of 1:300/400 births. This condition can result in exercise intolerance, shortness of breath, and chest pain. It may also result in labored breathing during exercise and overall loss of stamina. The current practice to correct this deformity is called the minimally-invasive Nuss procedure (NP), in which a surgeon takes a stiff metal bar and weaves it through the patient's rib cage and underneath the sternum. The bar is bent to match the shape of the patient's rib cage and rotated into its final position inside the chest cavity. This procedure typically produces instant correction of the deformed sternum, which correction distance far surpasses the region of non-painful skeletal deformation. The bar is placed between the ribs in such a way that one rib on either side of the sternum provides a vertical support to the bar, and the reaction load from the displaced sternum is effectively shifted to the ribs. The ends of the bar are often sutured to the outer ribs in efforts to create a fixed connection between the bar and the patient's body, which helps prevent flipping and jostling of the bar.

Because the NP typically produces complete and immediate correction, in many cases, it also causes extreme pain. Healthcare providers traditionally counteract this side-effect by prescribing opiates; because of the increased chance for opiate addiction, an alternative solution is desired. Additionally, some patients may entirely forgo the operation because of the anticipated pain following the operation.

The illustrative device of the present disclosure, or a linear ratchet bar, is comparable in size to the Nuss bar and is configured to be inserted into the patient's body using a similar procedure. The illustrative linear ratchet bar reduces patient pain by extending the PE correction stage to a longer period through a more gradual correction. In addition, the linear ratchet bar of the present disclosure is more flexible than that of the bar used in the NP. This provides easier implantation, leading to a less dangerous surgical procedure.

According to an illustrative embodiment of the present disclosure, a linear ratchet bar for pectus excavatum repair includes a first end portion extending between a proximal end and a distal end, a second end portion in spaced relation to the first end portion, the second end portion extending between a proximal end and a distal end, and a flexible center portion connecting the distal end of the first end portion and the distal end of the second end portion. The center portion is moveable between an undeflected state and a deflected state. The center portion extends outwardly in a convex manner from the distal end of the first end portion and the distal end of the second end portion in the undeflected state, and the center portion extends inwardly in a concave manner from the distal end of the first end portion and the distal end of the second end portion in the deflected state.

According to an illustrative embodiment of the present disclosure, a linear ratchet bar further includes a first ratchet device operably coupled to the first end portion and the center portion, and a second ratchet device operably coupled to the second end portion and the center portion. Each of the first ratchet device and the second ratchet device includes a plurality of teeth and a pawl arm supporting a pawl. In the deflected state, the pawl is in contact with the center portion and slidably engages successive teeth of the ratchet device as the center portion moves from the deflected state to the undeflected state. The pawl then advances over the teeth of the ratchet device, locking the center portion in place and preventing movement back to a previous deflected state.

According to another illustrative embodiment of the present disclosure, a system for pectus excavatum repair includes a linear ratchet bar. The linear ratchet bar includes a first end portion extending between a proximal and a distal end, and a second end portion in spaced relation to the first end portion. The second end portion extends between a proximal end and a distal end. The linear ratchet bar further includes a flexible center portion connecting the distal end of the first end portion and the distal end of the second end portion. The center portion is moveable between an undeflected state and deflected state. The center portion extends outwardly in a convex manner from the distal end of the first end portion and the distal end of the second end portion in the undeflected state, and the center portion extends inwardly in a concave manner from the distal end of the first end portion and the distal end of the second end portion in the deflected state. The system further includes a pulling device that may be placed on a patient. Upon actuation, the pulling device increases the force that the center portion exerts to move from the deflected state to the undeflected state. Increasing the force that the center portion exerts assists in aiding the linear ratchet bar from a deflected state to an undeflected state.

According to a further illustrative embodiment of the present disclosure, a method of correcting pectus excavatum includes the step of providing a linear ratchet bar. The linear ratchet bar includes a first end portion, a second end portion in spaced relation to the first end portion, a flexible center portion connecting the first end portion and the second end portion, and a ratchet device supported by the center portion including a pawl that engage a plurality of teeth. A further step includes inserting the elastic bar within a chest cavity of a patient, ensuring the first end portion and the second end portion engage opposing ribs, and the flexible center portion engages a sternum of the patient. Next, the flexible center portion applies force against the sternum in an outward direction as the flexible center portion moves from a deflected position to an undeflected position. Lastly, as the flexible center portion moves from a deflected position to an undeflected position, the pawls advance successively over the of teeth of the ratchet device, locking the flexible center portion and preventing movement back to a previous deflected state.

Additional features and advantages of the present disclosure will become apparent to those skilled in the art upon consideration of the following detailed description of the illustrative embodiments exemplifying the disclosure as presently perceived.

### BRIEF DESCRIPTION OF THE DRAWINGS

The detailed description of the drawings particularly refers to the accompanying figures in which:

FIG. 1 is a perspective view of an illustrative linear ratcheting bar of the present disclosure;

FIG. 2 is diagrammatic side view of the linear ratchet bar of FIG. 1, showing the bar in a deflected state against a depressed sternum and the corresponding force in order to move the bar into a undeflected final state in which the position of sternum has been corrected;

FIG. 2A is a detail view of the ratchet device of FIG. 2;

FIG. 3 is a perspective view of an illustrative pawl arm independent from the linear ratchet bar;

FIG. 4 is another perspective view of an illustrative pawl arm;

FIG. 5 is a detail view of the pawl held at the end of the pawl arm of FIG. 4;

FIG. 6 is a side view of an illustrative pawl arm;

FIG. 7 is side view of an alternative embodiment pawl arm;

FIG. 8 is a detail perspective view of the ratcheting portion of an alternative embodiment linear ratchet bar;

FIG. 9 is a detail side view of the ratcheting portion of an alternative embodiment linear ratchet bar;

FIG. 10 is a detail side view showing a pawl engaging an embodiment of the ratchet teeth; and

FIG. 11 is a detail side view showing a pawl engaging an alternative embodiment of the ratchet teeth.

### DETAILED DESCRIPTION OF THE DRAWINGS

For the purposes of promoting an understanding of the principles of the present disclosure, reference will now be made to the embodiments illustrated in the drawings, which are described herein. The embodiments disclosed herein are not intended to be exhaustive or to limit the invention to the precise form disclosed. Rather, the embodiments are chosen and described so that others skilled in the art may utilize their teachings. Therefore, no limitation of the scope of the claimed invention is thereby intended. The present invention includes any alterations and further modifications of the illustrated devices and described methods and further applications of principles in the invention which would normally occur to one skilled in the art to which the invention relates.

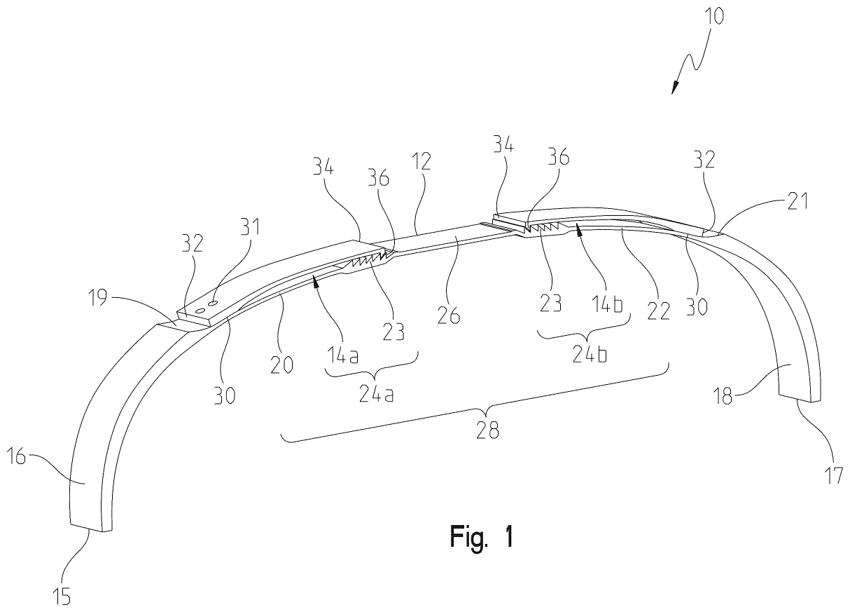

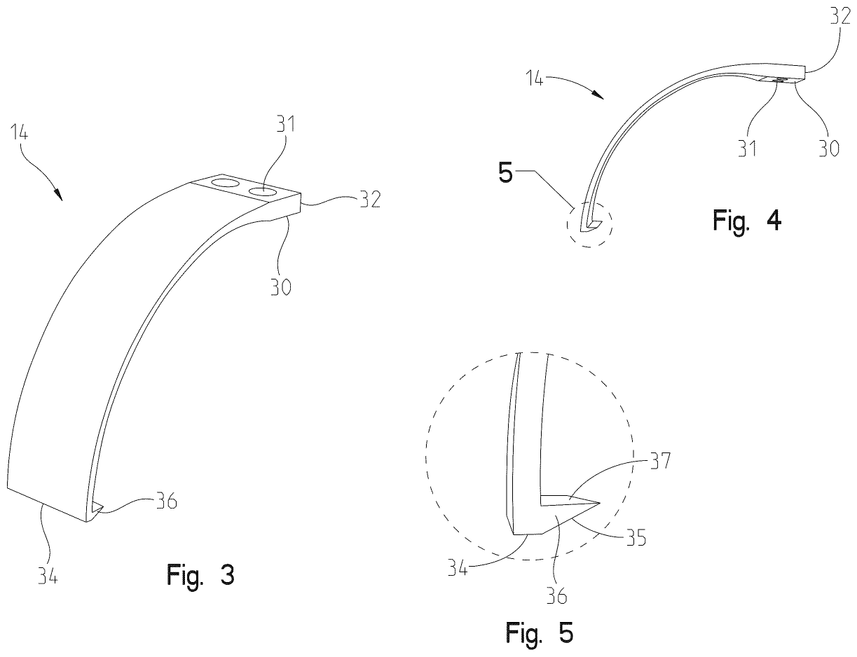

Referring initially to FIGS. 1-4, an illustrative linear ratchet bar 10 of the present disclosure is shown and configured to correct a pectus excavatum deformity in a human patient and do so in a gradual, iterative process. The illustrative bar 10 includes a unitary body 12 having an arcuate shape. Affixed to the body 12 are first and second pawl arms 14a and 14b that have a complimentary arcuate shape. The pawl arms 14a and 14b may be affixed to the body 12 in spaced relation to each other with basic hardware (i.e. screws and nuts), welding, or by other manufacturing processes familiar to those skilled in the art.

It can be recognized that the body 12 and the pawl arms 14a and 14b may be formed of a biocompatible material, such as Ti-6Al-4V (Ti-64), or any such biocompatible material with similar stiffness and yield strength properties. The body 12 and the pawl arms 14a and 14b may be fabricated using wire electrical discharge manufacturing (“EDM”) or any similar precision manufacturing method. Further, the bar 12 and the pawl arms 14a and 14b could be covered in a biocompatible silicon sheath, or a material of similar properties that could protect the bar from tissue ingrowth.

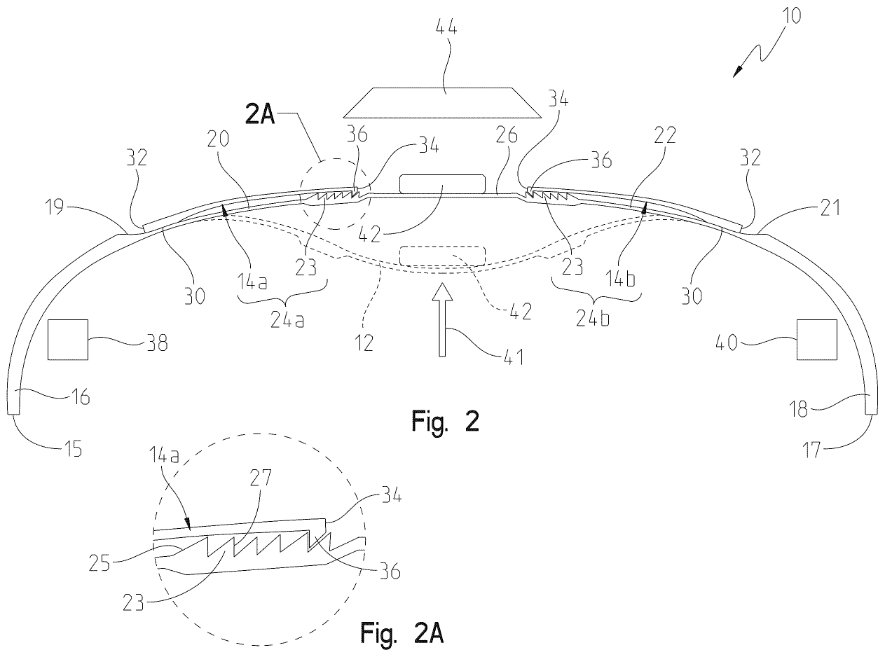

Referring further to FIGS. 1 and 2, the body 12 illustratively includes a first end portion 16 and a second end portion 18. The first end portion 16 has a proximal end 15 and a distal end 19. Likewise, the second end portion 18 has a proximal end 17 and a distal end 21. Illustratively, the first pawl arm 14a is coupled to the first end portion 16 proximate the distal end 19, and the second pawl arm 14b is coupled to the second end portion 18 proximate the distal end 21. A center portion 28 extends between the distal ends 19 and 21 of the first end portion 16 and the second end portion 18, respectively. The end portions 16 and 18 are configured to perform a stabilization function, while the center portion 28 is configured to perform a correction function.

In one illustrative embodiment, the end portions 16 and 18 have a stiffness greater than the center portion 28. This may be accomplished due to the distal ends 19 and 21. As there is a transition from the end portions 16 and 18 to the center portion 28, the distal ends 19 and 21 provide a point where the material becomes less thick than the end portions 16 and 18. The bar 10 retains overall dimensions similar to the Nuss bar, including the overall width, overall height, and overall length. However, the conventional Nuss bar has a constant cross section and corresponding uniform stiffness, while the illustrative bar 10 includes the flexible center portion 28.

Still referring to FIGS. 1-4, the center portion 28 includes a first side 20 and a second side 22, meeting together at a center 26. Flanking either side of the center 26 are first and second ratchet devices 24a and 24b. The ratchet devices 24a, 24b include a plurality of teeth 23. As seen in FIG. 2A, each individual tooth 23 features a sliding surface 25 and a locking surface 27. Coupled to the center portion 28 are at least two pawl arms 14a and 14b. In one illustrative embodiment, the pawl arms 14a and 14b have a mating surface 30 configured to sit flush on the first and second side 20 and 22. As shown in FIG. 1, each pawl arm 14a, 14b may be mated to the body 12 by using screws 31. Alternative methods of attaching the pawl arms 14a and 14b may include welding, gluing, additive manufacturing or any other methods known to those who are skilled in the art. The mating surface 30 abuts is adjacent to a first edge 32 of the pawl arm 14. The pawl arm 14a, 14b then extends from the first edge 32 to a second edge 34 that supports a pawl 36. The pawl engages the teeth 23, or alternatively, is in contact with the first or second side 20 and 22 of the center portion 28.

FIG. 2 diagrammatically illustrates the linear ratchet bar 10 placed in a skeletal model of a human rib cage. More particularly, FIG. 2 shows the first and second end portions 16 and 18 resting on ribs 38 and 40 for support. In a deflected state, denoted by the dotted lines in FIG. 2, the body 12 applies the outward force 41 on the sternum 42. The force 41 applied to the sternum 42 lessens the stiffness of the chest wall, gradually corrects the position of the sternum 42, and returns the bar 10 to its undeflected state.

A pulling device 44 may be employed to supplement the force 41 to the sternum 42. Illustratively, the pulling device may be a suction device, such as a bell vacuum or any like device known to those who are skilled in the art.

FIG. 5 is a detail view of the illustrative pawl 36 supported on the second edge 34. The pawl 36 features a sliding edge 35 and a locking edge 37. As the pawl 36 engages the ratchet device 24 and returns to an undeflected state, the sliding edge 35 moves along the sliding surface 25. Once the pawl 36 has advanced along the entire length of the sliding surface 25, the pawl 36 drops onto the next tooth 23 and the locking edge 37 abuts the locking surface 27, preventing the pawl 36 from regressing.

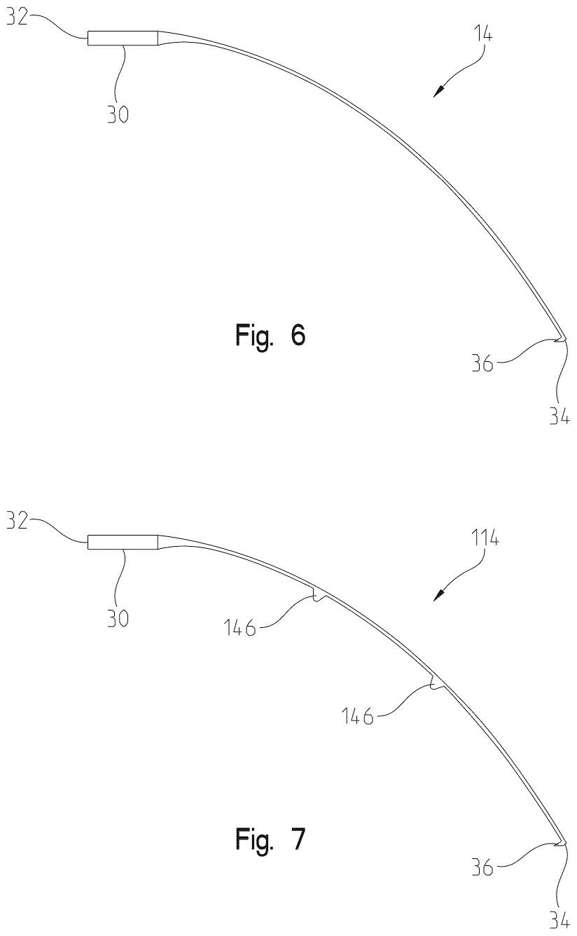

FIG. 6 shows a side view of the illustrative pawl arm 14. The pawl arm 14 is configured to be pre-bent to a smaller diameter than that of the body 12 as means to keep the pawl arm 14 in consistent contact with the body 12. FIG. 7 shows an alternative embodiment of a pawl arm 114 that includes a plurality of supports 146 that encourage the preferred consistent contact with the body 12.

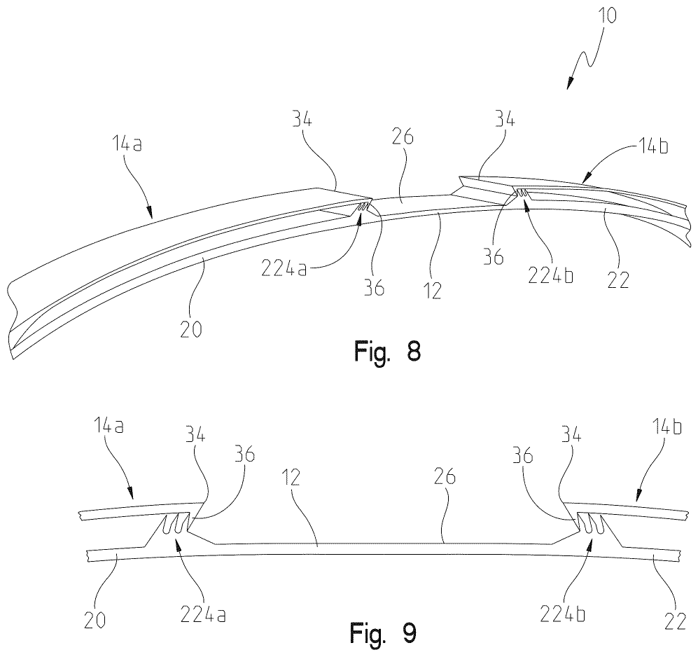

FIGS. 8 and 9 show the linear ratchet bar 10 but illustratively feature an alternative embodiment of the ratchet devices 224a and 224b. The illustrative ratchet devices 224a and 224b are compatible with the linear ratchet bar 10 as previously described herein, and as such similar components are identified with like reference numbers.

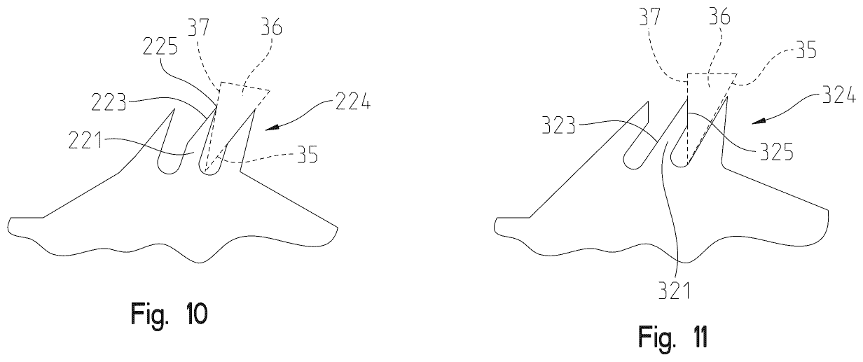

FIG. 10 is an illustrative embodiment of the ratchet device 224 engaging the pawl 36 of respective pawl arm 14a, 14b. A tooth 221 has a sliding surface 223 and a locking surface 225. The corresponding sliding edge 35 of the pawl 36 slides along the sliding surface 223 as the bar 10 relaxes into an undeflected state. Once the end of the sliding surface 223 is met, the pawl 36 moves to the next tooth 221 and the locking edge of the pawl 36 engages the locking surface 225 of the tooth, preventing the bar 10 from deflecting past the tooth 221 that has been advanced upon.

Likewise, FIG. 11 is an illustrative embodiment of the ratchet device 324 engaging the pawl 36. A tooth 321 has a sliding surface 323 and a locking surface 325. The corresponding sliding edge 35 of the pawl 36 slides along the sliding surface 323 as the bar 10 relaxes into an undeflected state. Once the end of the sliding surface 323 is met, the pawl 36 moves to the next tooth 321 and the locking edge of the pawl 36 engages the locking surface 225 of the tooth, preventing the bar 10 from deflecting past the tooth 321 that has been advanced upon.

An illustrative method for correcting pectus excavatum may include a surgical procedure in which the linear ratchet bar 10 of the present disclosure is weaved into a patient's ribcage such that the center 26 is placed posteriorly to the sternum 42. End portions 16 and 18 rest on support ribs 38 and 40. When implanted, the center portion 28 of the bar 10 deflects under the load of the sternum 42. The load created by the sternum 42 is applied to the support ribs 38 and 40 whereas the deflected bar 10 applies an opposite force 41 on the sternum 42 as it returns to an undeflected state. In the deflected state, the forces applied resemble three-point bending.

The pawl arms 14a, 14b are illustratively configured to be bent into an arc with a smaller diameter than the body 12 of the bar 10. As such, the pawl 36 remains in contact with either the first or second side 20 and 22 of the center portion 28 or the ratchet device 24 when in the deflected state or the undeflected state, respectively.

Once the bar 10 is placed, the force 41 applied on the sternum 42 eventually decreases the stiffness of the chest wall, subsequently correcting the position of the sternum 42 as the bar 10 relaxes into its undeflected state. During this relaxation, the pawls 36 advance from the first and second side 20 and 22 of the center portion 28 to the first tooth 23 of the ratchet device 24. The sliding edge 35 of the pawl 36 moves along the sliding surface 25 of the first tooth 23 of the ratchet device 24. Once the pawl 36 has advanced along the entire length of the sliding surface 25, the pawl drops onto the next successive tooth 23 and the locking edge 37 abuts the locking surface 27, preventing the pawl 36 from regressing. This step repeats until the pawl 36 has progressed over every tooth 23 of the ratchet device 24.

If it is deemed necessary due to any perceived difficulties, a pulling device 44 (e.g., a vacuum bell) may be employed for use to increase the force 41 applied to the sternum 42, pulling the chest wall in the direction of the force 41, helping to advance the pawl 36. Alternatively, a pulling device 44 may be employed as part of the standard surgical and procedural process. The advancement of the pawl 36 over the last tooth 23 of the ratchet device 24 coincides with the return of the bar 10 into its undeflected state, at which point the position of the sternum 42 has been corrected and the bar 10 may be removed.

Although the invention has been described in detail with reference to certain preferred embodiments, variations and modifications exist within the scope of the invention as described and defined in the following claims.

What is claimed is:

1. A linear ratchet bar for pectus excavatum repair, the linear ratchet bar comprising:
a first end portion extending between a proximal end and a distal end;
a second end portion in spaced relation to the first end portion, the second end portion extending between a proximal end and a distal end;
a flexible center portion connecting the distal end of the first end portion and the distal end of the second end portion, the center portion being moveable from a undeflected state to a deflected state, and vice versa, the center portion extending outwardly in a convex manner from the distal end of the first end portion and the distal end of the second end portion in the undeflected state, and the center portion extending inwardly in a concave manner from the distal end of the first end portion and the distal end of the second end portion in the deflected state;
a first ratchet device operably coupled to the first end portion and the center portion;
a second ratchet device operably coupled to the second end portion and the center portion;
wherein each of the first ratchet device and the second ratchet device includes:
a plurality of teeth; and
a pawl arm supporting a pawl;
wherein in the deflected state the pawl is in contact with the center portion and slidably engages successive teeth of the ratchet device as the center portion moves from the deflected state to the undeflected state; and
wherein the pawl advances over the teeth of the ratchet device, locking the center portion in place and preventing movement to a previous deflected state.

2. The linear ratchet bar of claim 1, wherein first end portion, the second end portion and the flexible center portion are integrally formed of a biocompatible material.

3. The linear ratchet bar of claim 1, wherein the first end portion and the second end portion are stabilization members, and the center portion is a correction member.

4. The linear ratchet bar of claim 3, wherein the first end portion and the second end portion each have a stiffness greater than the flexible center portion.

5. The linear ratchet bar of claim 1, wherein the pawl arm is bent to a smaller diameter than that of the center portion.

6. The linear ratchet bar of claim 5, wherein at least two supports are operably coupled to the pawl arm and equally spaced in relation to one another.

7. The linear ratchet bar of claim 1, wherein the teeth further include a sliding face and a locking face.

8. The linear ratchet bar of claim 7, wherein the pawl further includes a sliding edge and a locking edge wherein as the pawl advances over successive teeth, the sliding edge of the pawl slidably moves along the length of the sliding face of the teeth until the pawl reaches the end of the sliding face and the locking edge of the pawl engages the locking face of the teeth.

9. A system for pectus excavatum repair, the system comprising:
a linear ratchet bar including:
a first end portion extending between a proximal and a distal end;
a second end portion in spaced relation to the first end portion, the second end portion extending between a proximal end and a distal end;
a flexible center portion connecting the distal end of the first end portion and the distal end of the second end portion, the center portion being moveable from a undeflected state to a deflected state, and vice versa, the center portion extending outwardly in a convex manner from the distal end of the first end portion and the distal end of the second end portion in the undeflected state, and the center portion extending inwardly in a concave manner from the distal end of the first end portion and the distal end of the second end portion in the deflected state; and
a pulling device;
wherein the pulling device may be placed externally of the linear ratchet bar and upon actuation increases the force that the center portion exerts to move from the deflected state to the undeflected state; and
wherein increasing the force that the center portion exerts assists in aiding the linear ratchet bar from a deflected state to an undeflected state.

10. The system of claim 9, wherein the pulling device comprises a vacuum bell for pulling a vacuum.

11. The system of claim 9, wherein first end portion, the second end portion and the flexible center portion are integrally formed of a biocompatible material.

12. The system of claim 9, wherein the first end portion and the second end portion are stabilization members, and the center portion is a correction member.

13. The system of claim 12, wherein the first end portion and the second end portion each have a stiffness greater than the flexible center portion.

14. The system of claim 9, the linear ratchet bar further including:
a plurality of ratchet devices, the ratchet devices including:
a plurality of teeth; and
a plurality of pawls supported by pawl arms, the pawl arms coupled to the flexible center portion;
wherein in the deflected state the pawl is in contact with the center portion and slidably engages successive teeth of the ratchet device as the center portion moves from the deflected state to the undeflected state.

15. The system of claim 14, wherein the pawl arm is bent to a smaller diameter than that of the center portion.

16. The system of claim 15, wherein at least two supports are operably coupled to the pawl arm and equally spaced in relation to one another.

17. The system of claim 14, wherein the teeth further include a sliding face and a locking face.

18. The system of claim 17, wherein the pawl further includes a sliding edge and a locking edge wherein as the pawl advances over successive teeth, the sliding edge of the pawl slidably moves along the length of the sliding face of the teeth until the pawl reaches the end of the sliding face and the locking edge of the pawl engages the locking face of the teeth.

19. A method of correcting pectus excavatum, the method comprising the steps of:
providing a linear ratchet bar including a first end portion, a second end portion in spaced relation to the first end portion, a flexible center portion connecting the first end portion and the second end portion, and a ratchet device supported by the center portion including a pawl that engage a plurality of teeth;
inserting the linear ratchet bar within a chest cavity of a patient, wherein the first end portion and the second end portion engage opposing ribs, and the flexible center portion engages a sternum of the patient; and
wherein the flexible center portion applies force against the sternum in an outward direction as the flexible center portion moves from a deflected position to an undeflected position; and
wherein as the flexible center portion moves from a deflected position to an undeflected position, the pawls advance successively over the of teeth of the ratchet device, locking the flexible center portion and preventing movement back to a previous deflected state.

20. The method of claim 19, wherein the first end portion and the second end portion each have a stiffness greater than the flexible center portion.
